(19) United States
(12) Patent Application Publication
(10) Pub. No.: US 2006/0140955 A1
(43) Pub. Date: Jun. 29, 2006

(54) Specific isotype antibodies of secretion-excretion anti-antigens of leishmania sp of promastigote or amastigote forms used as protection, resistance and curing markers of mammals to leishmaniases and to intracellular pathogenic micro-organism infections, and as immunotherapeutic effectors

(75) Inventors: Gerard Papierok, Hyeres (FR); Serge Vicens, Gignac la Nerthe (FR); Jean-Loup Lemesre, Montpellier (FR)

Correspondence Address: EGBERT LAW OFFICES, 412 MAIN STREET, 7TH FLOOR, HOUSTON, TX, 77002, US

(73) Assignee: BIO VETO TESTS, en abrege BVT (SAS), F-83500 La Seyne Sur Mer (FR)

(21) Appl. No.: 10/521,922

(22) PCT Filed: Jul. 25, 2003
(86) PCT No.: PCT/FR03/02358

§ 371 Date: Aug. 29, 2005

(30) Foreign Application Priority Data
Jul. 26, 2002 (FR) 02/09506

(51) Int. Cl. A61K 39/395

(52) U.S. Cl. 424/151.1; 530/388.6

(57) ABSTRACT

The invention concerns immunoglobulins characterized in that they include immunoglobulins of IgG₂ classes and corresponding sub-classes, specific of secretion-excretion antigens of the prosmatigote and asmatigote forms of Leishmania sp, capable of lyzing Leishmania sp amastigotes and promastigotes in vitro, and neutralizing their proliferation.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention involves the detection and the use of IgG2 isotype antibodies specific to secretion-excretion antigens of Leishmania which enable:
proof of a cell-mediated immunity depending on the T lymphocytes of the Th1 type,
monitoring of the immune response after vaccination or treatment,
evaluation of the efficacy of a chemotherapeutic and/or immunotherapeutic treatment in mammals and in particular, in humans, canines, felidae, and equidae,
evaluation of a state of resistance to leishmaniasis,
establishment of an immunotherapy.

The present invention also involves:
proof of a specific humoral response represented by the IgG of isotype IgG₂ in dogs and/or other isotypes in other mammals, a humoral response associated with a cell-mediated response depending on the T lymphocytes of the Th1 type,
the detection of these IgG specific to the parasite Leishmania sp using the well-defined secretion-excretion antigens and
the neutralizing role of these IgG characterized by the inhibition of the proliferation of the amastigote or promastigote forms of Leishmania.

### BACKGROUND OF THE INVENTION

The therapeutic vaccine complex comprised of secretion-excretion products in an axenic and serum-free medium, defined as described in the French patent number 01/07606 dated Jun. 11, 2001, induces an immunostimulation of the lymphocytary system of the Th1 type which ensures the establishment of a protective immunity. This cell-mediated immunity, which is characterized by an activation of the lymphocytes and macrophages and by the synthesis of cytokines specific to the Th1 type, is linked to a humoral immunity characterized by the production of specific IgG which neutralize certain isotypes.

In a very simple way, it is possible to divide the immune responses into two large qualitatively distinct categories: humoral responses that introduce the production of antibodies by the B lymphocytes, and cellular responses (delayed hypersensitivity reaction, cytotoxic reactions) for which the effective cells are the T lymphocytes. It seems that in the majority of experimental models and clinical situations studied, the cytokines having a lymphocytary origin are essentially produced by the auxiliary T cells CD4+ (or helpers) whose role is to modulate or regulate the humoral and cellular immunity and which recognize the antigen in association with molecules of class II of the major histocompatibility complex. A major concept emerged in 1985 when T. Mosmann and R. Coffman proposed that the T lymphocytary cells CD4+ expressing the auxiliary functions were in fact heterogens. Thus, via the study of T lymphocytary clones CD4+ of mice, cultivated over a long term, these authors described the existence of two major sub-populations that can be distinguished via their secretion profile from cytokines, i.e. the Th1 cells (for type 1 T helpers) and Th2 cells (for type 2 T helpers).

Take, for example, leishmaniasis, which is a parasitic infection endemic, or even epidemic, in tropical and subtropical regions of the world. Leishmania, flagellate protozoans of the family Trypanosomatidae and genus Leishmania, are the pathogenic agents responsible for these diseases.

Numerous studies concerning the immune responses during experimental murine leishmaniases have led to demonstration of the predominant role of cell-mediated immunity and the existence of a duality of the immunological response. There are fundamentally two types of responses against leishmanias: one is described by the sensitivity, the other is described by the resistance. The different subpopulations of T lymphocytes (CD4+) limit or exacerbate the infection by means of the lymphokines they secrete. It has thus been demonstrated that the subpopulation of auxiliary T lymphocytes of the Th1 type (producer of interferon gamma and interleukine 2) was capable of eliminating the amastigote intracellular forms by means of the activation of macrophages (Reiner S.L et al., Annu Rev Immunol, 1995, 13, 151-177. Review). Conversely, the subpopulation of auxiliary T lymphocytes of the type Th2 (producer of interleukine 4) is responsible for exacerbating the disease.

In humans, certain facts are comparable by nature. In the dog (natural “reservoir” receptive host in the life cycle of L. infantum), the duality of the immunological response is likely. Only one study led by Pinelli et al. (Infect. Immun., 62:229, 1994) on animals experimentally and naturally infected by L. infantum made it possible to show that the asymptomatism of the dog (clinical state frequently encountered) is accompanied by the absence of a humoral response and the development of a cell-mediated immunity of the Th1 type with a hypersensitivity reaction of the positive delayed type and elevated rates of interleukine 2 and TNF-α circulating in the biological liquids.

The polarization of the immune responses to a Th1 or Th2 phenotype has been associated with numerous pathological situations.

For infections by Leishmania, Trypanozoma, Candida and other intracellular organisms such as Mycobacterium and Listeria, a Th1 response correlates to resistance to the pathogen.

### BRIEF SUMMARY OF THE INVENTION

The present invention consists notably in detecting antibodies whose production accompanies the polarization of a cell-mediated immunity to a Th1 type.

According to specialists like Pinelli (Pinelli. E et al. Infect. Immun, 1994, 62: 229-235) leishmanian dogs correspond to type Th2 activation of the lymphocytary system having an elevated antibody response. This increased antibody production corresponds to hyperproteinemia and induces the appearance of immune complexes that cause a renal problem (increase in creatinine and blood urea). On the other hand, it has been demonstrated (RIERA, C. et al, vet parasitology, 1999, 84, 1-2, p. 33-47) that the rate of total IgG decreased in experimentally infected dogs after treatment with glucantime; however, this treatment only induces a temporary recession of symptoms. Relapses remain possible and are not predictable.

Nevertheless, certain preliminary work in humans (KAWANO. P et al, Parasite Immunol, 1995, 17, 451-458) and in dogs (NIETO C. G. et al, Vet Immunol and Immunopathology, 1999, 67, 117-130) shows that the IgG isotypes would be markers of the Th1/Th2 immunitary dichotomy. In addition, some authors (DEPLAZES. P et al, Parasite Immunology, 1995, 17, p. 451-458 /RIERA, C. et al, vet parasitology, 1999, 84, 1-2, p. 33-47) have studied more specifically the importance of the ratio of isotypes IgG₁/IgG₂ in leishmanian dogs before and after treatment. These IgG₁/IgG₂ were specific to a total somatic antigen of the promastigote, the rate of IgG₂ remained constant before and after treatment; on the other hand, a significant drop of IgG₁ was noticed after treatment.

However, the specificity of these isotypes to a well-defined antigen or epitope has never been established as the role of these isotypes.

Dogs that received the therapeutic vaccine complex described in the French patent 01/07606 cited above and thus protected, have significant rates of IgG₂ specific to secretion-excretion proteins, which is compliant with the preferential expansion of T lymphocytes of the Th1 type. On the contrary, the dogs that received a placebo as well as the leishmanian dogs do not have the specific IgG₂.

The secretion-excretion antigens of the protozoan Toxoplasma gondii (patent EP 0 301 961 A) have been studied as vaccines and therapeutic products, but these antigens have never been correlated to the IgG of a particular isotype having a well defined role. On the other hand, some authors (CIBRELUS. P et al, Parasite, 1999, 6 no. 2, p121-129) have shown that naturally infected dogs have total IgG towards the secretion-excretion antigens of Leishmania infantum but also no correlation has been established with immunoglobulins of the specific isotype having a well defined role.

The present invention also has the purpose of showing that the immunoglobulins of the IgG₂ classes and corresponding sub-classes, linked to the Th1 system, are specific to a well-defined antigen: the secretion-excretion antigens of Leishmania (ES), and have a neutralizing action towards the proliferation of amastigotes and promastigotes of Leishmania sp.

These specific IgG₂ induced in dogs immunized with secretion-excretion antigens do not exist in naturally infected mammals. They are thus different from the ones cited in the “Riera” and “Deplazes” documents mentioned above which describe the IgG of naturally leishmanian dogs.

These IgG₂ are, more precisely, specific to a major protein excreted-secreted by the 2 stages of Leishmania sp of the family of the PSA “Protein Surface Antigens” corresponding to a range of molecular mass from 52 to 58 Kda. This major immunogen has an “immunologically silent” epitope during an infection by leishmanias in humans or in dogs. This epitope located in the carboxyterminal preserved region is recognized by the IgG in dogs. Some authors (KEMP. M et al, FEMS Immunol and med microbiology, Netherlands, 1998, 20 no. 3, p 209-218) have demonstrated the existence of a glycoprotein of surface PSA-2 in Leishmania major responsible for cutaneous leishmaniasis in humans and in mice. This glycoprotein that corresponds to a constituent surface antigen of the promastigote form of the parasite and not of excretion-secretion antigens, induces an immunitary state of the Th1 type in mice. Other teams (JIMENEZ-RUIZ. A et al, Europ J of Biochemistry/FEBS Germany, 1998, 25 no. 1-2, p 389-397) demonstrate that a PSA of a promastigote of Leishmania infantum is a major immunogen of the B cells in naturally infected dogs. In the two cases, the specificity of IgG₂ having a neutralizing activity has not been proven. Moreover, the antibody response during a natural infection according to JIMENEZ-RUIZ. A et al. is essentially directed against the repeated patterns rich in Leucine, and not against the carboxyterminal part, the major antigen of the surface of Leishmania infantum (and thus not of an excretion-secretion antigen).

The major antigen of the excretion-secretion products has been characterized by the elaboration of monoclonal antibodies (AMC), and it belongs to the family of the PSA and corresponds to the range of molecular mass from 52 to 58 Kda. Two banks of ADNc expression of the promastigote forms and amastigote forms of Leishmania have been immunologically separated using AMC F5 in order to identify the major immunogens of the AES. This has made it possible to isolate ADNc from a given clone which codes an excreted-secreted protein of the PSA family.

These PSA proteins have characteristic repeated domains rich in leucine.

The analysis of ADNc sequences reveals a peptide sequence corresponding exactly to the COOH-terminal end of the isolated PSAs (see FIG. 1: schematic representation of the COOH terminal end of PSAs identified by the monoclonal antibody F5).

Using a recombinant protein coding for the carboxyterminal part of the PSAs, it has been shown that patients or dogs which have contracted leishmaniasis are incapable of producing antibodies against the epitopes carried by the recombinant protein (see FIG. 2: Analysis of the humoral response of patients and dogs naturally infected with regard to the recombinant protein 6 (His)-COOH-PSA), while they produce antibodies against native ES antigens, thus against other epitopes present on the native PSAs. On the contrary, dogs immunized with AESs of promastigotes of Leishmania infantum, and protected against visceral leishmaniasis, have antibodies of the isotype IgG2 specific to the carboxyterminal part (see FIG. 3: Analysis of the humoral response of dogs immunized by the AESs of promastigotes of Leishmania infantum relative to the recombinant protein 6 (His)-COOH-PSA).

The present invention also has the purpose of showing that after chemotherapy or immunotherapy, the IgG2s in dogs specific to the ES antigens of Leishmania and notably of the carboxyterminal part of PSA appear with significant improvement in the general state of dogs that have contracted Leishmaniasis.

### DETAILED DESCRIPTION OF THE INVENTION

As for all vector-transmitted diseases, leishmaniases are characterized by a life cycle that is relatively simple since it is divided between two hosts, mammals and insects (phlebotomes), and consists of two main forms:
a flagellate form called a promastigote, present in the digestive tract of the phlebotomic vector, where it multiplies prior to acquiring its form that is infectious for the mammalian host, also called the metacyclic form;
a non-flagellate form called amastigote, present in the mammalian host such as the dog and human.

In order to study the synthesis specific to the antibody IgG₂ in dogs, 2 types of experiments were performed after immunization by the therapeutic vaccine complex comprised of ES: role of the in vitro effect of the immune serum of dogs on the kinetics of multiplication of the different stages of the parasite Leishmania infantum; dosage of the IgG₂ specific excretion-secretion anti-antigens of Leishmania.

For our experiments, we used the vaccine complex composed of excretion-secretion antigens of promastigotes noted in the French patent application no. 01/07606 mentioned above.

The dogs are immunized according to the following scheme:

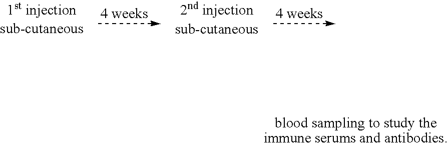

Another type of experiment was performed: dosage of the specific IgG2 anti antigens ES of Leishmania in leishmanian dogs treated by chemotherapy. For this experiment, we used, as the antigen, the recombinant protein coding for the carboxyterminal part of PSA.

The methods used are the following:

a. Parasitic Material:

The culture of promastigotes of leishmania infantum MON. 1 is collected in a stationary phase of growth. The parasites are washed 3 times in 15 ml of sterile PBS (centrifugation at 4° C. for 5000 revolutions over 10 min) then collected in 1 ml of PBS.

The promastigote/amastigote differentiation and thus the multiplication of the amastigotes requires 0.5×10⁶ parasites per ml of culture medium MAA20 pH 5.8 in a culture chamber incubated at 37° C. and under 5% CO2 (conditions representing the phagolysosomial environment); the proliferation of promastigotes requires 10⁶ parasites per ml of culture medium RPMI 20% pH 7.2 in a culture chamber incubated at 25° C. (conditions representing the phlebotome).

A viability test with Trypan® (Blue at 0.4% in PBS and a Thomas cell counting must be done in order to resuspend the parasites in sterile PBS so that they are at 10⁶/ml.

b. Preparation of the Serums:

One aliquot of each serum is decomplemented by passage through a water bath for 45 min. at 56° C. then centrifuged 12 min for 12000 revolutions in order to prevent agglutinins.

First, with the serumal ratio in antibody not being known, a range of aleatory dilution is performed. For 10⁶ parasites, it is necessary to put it in contact with 20 μl of serum:
1^{st} test pure serum: 5.10⁶ parasites (divided into RPMI and MAA) i.e. (5.10⁶×20)/10⁶=100 μl of pure serum.
2^{nd} test dilution to ½: 50 μl of pure serum +50 μl of sterile PBS buffer.
3^{rd} test dilution to ¼: 25 μl of pure serum +75 μl of sterile PBS.
4^{th} test dilution to ⅛: 12.5 μl of pure serum +87.5 μl of sterile PBS

c. Contact Promastigotes-Antibody:

The contacts are made in 1.5 ml Eppendorff tubes where 5.10⁶ parasites are treated with 100 μl of serum for 30 min. over ice (minimum time necessary for a promastigote to infect a macrophage). The excess antibody is then washed twice with 1 ml of PBS and by a centrifugation for 5000 revolutions for 10 min., repeated twice.

d. Analyses:

One aliquot of 10 ul for each test is then taken immediately after contact in order to perform a viability test with Trypan® Blue and a Thomas cell counting, the surplus serving for the cultivation from which a counting is done daily.

Proliferation of the Promastigotes (Medium: RPMI 20%)

A Thomas cell counting from each test is done daily in order to monitor the kinetics of multiplication.

From an aliquot of each cultivation test, a parasitic smear fixed with methanol then colored with May-Grunvald-Giemsa is done daily, whereby the percentage of the promastigote form, spheromastigote form and amastigote form is determined by counting 100 parasites.

Proliferation of the Amastigotes (Medium: MAA 20)

From a second aliquot of each cultivation test, a Thomas cell counting is done in order to monitor the kinetics of multiplication.

The calculations of inhibition of growth are done on the following model:

The number of parasites treated with the healthy serum (=Ns)→100% of growth

The number of parasites treated with the immunized serum (=Ni)→×% of growth

% inhibition=100 ×(Ni×100)/Ns

e. Studies of the Antibodies:

The total IgG antibodies in dogs anti Leishmania are detected by traditional immunofluorescence using slides coated with promastigotes (serological reference method for canine leishmaniasis).

The detection of the IgG2 specific to ESP is done by ELISA according to the technique of microtitration by Kweider et al (J. immunol, 1987, 138:299) using the ESP as antigens and a conjugated antilgG2.

f. Analyses Performed in Dogs Immunized with the ESP:

A complete inhibition study of the proliferation of amastigotes and promastigotes was done on two dogs:

The dog named MINON: Weimaraner breed, female, 4 years old, vaccinated with ESP

The dog named MUMA: Brittany Spaniel breed, female, 7 years old, control placebo

A viability test of the promastigotes was done on 6 other dogs in an experimental study and 4 dogs in a local environment study (test of the vaccine complex ES in the endemic zone) compared to 2 control dogs.

g. Study of Antibodies in Leishmanian Dogs treated by Chemotherapy:

For the 3^{rd} experiment, serums of 5 dogs infected with visceral leishmaniasis and treated by glucantime for 1 to 2 months, coming from Dr B, were analyzed.

h. Studies of Antibodies in Leishmanian Dogs Treated by Immunotherapy with ESP:

Three leishmanian dogs were treated by subcutaneous injection of ESP (3 injections of 50 μg, each injection two weeks apart).

The total antibodies IgG in dogs anti Leishmania were detected by immunofluorescence and the detection of antibodies IgG2 specific to the ESPs was done by ELISA as described above (see: e.: Study of antibodies) but using as an antigen the recombinant protein coding for the carboxyterminal part of the PSA.

Results:

1. Study of the Proliferation of Promastigotes of L. infantum:

The following study was done on parasites put into contact with a serum of a healthy dog or a dog immunized 4 times with the excretion-secretion products of promastigotes (ESP) and before challenge.

Determined by a test with Trypan® Blue, the viability of the promastigotes was 100% before contact with the serum. After 30 min. of contact, the viability with the healthy serum was always 100% at all dilutions, while with the serum immunized with ESP, it was only more than 50% with the pure serum, 73% with the serum diluted to ½, 92% and 94% with respectively the serum diluted to ¼ and ⅛ (see FIG. 4: Kinetics of proliferation of promastigotes after 30 minutes of contact with different dilutions of serums (Pure (A); ½ (B); ¼ (C); ⅛ (D)) in the healthy dog (S) Muma and immunized with the ESP(I) Minon).

The following table describes the development of the percentage of inhibition of the growth of promastigotes after contact of 30 minutes with serum of the immunized dog (ESP) relative to the serum of the healthy dog.

| | Percentage inhibition | | | | |
|---|---|---|---|---|---|
| | 1^{st} day | 2^{nd} day | 3^{rd} day | 4^{th} day | AverageI |
| pure/S pure | 93% | 92% | 98% | 73% | 89% |
| I ½/S ½ | 94% | 94% | 84% | 90% | 90% |
| I ¼/S ¼ | 79% | 60% | 45% | 0% | 46% |
| I ⅛/S ⅛ | ND | 81% | 90% | 81% | 84% |

Interpretation:

The inhibition of growth with the anti-ESP serum (the dog MINON) relative to the healthy serum approaches 90% regardless of the dose (aside from for the dilution ¼, manipulation errors?). It appears that the threshold value of activity ofthis serum is not obtained with this range of dilutions, at least the cytotoxic effect is powerful and the growth of promastigotes is greatly slowed down for all dilutions of the serums.

2. Study on the Proliferation of Amastigotes of L. infantum

The following study was done on cultures in MAA. 20 for differentiation into amastigotes, from promastigotes treated with a serum before challenge of a healthy dog or a dog immunized 4 times with the ESP (see FIG. 5: Kinetics of proliferation of amastigotes after 30 minutes of contact with different dilutions of serums (Pure (A); ½ (B); ¼ (C); ⅛ (D)) of the healthy dog (S) Muma and the dog immunized with ESP(I) Minon).

The following table indicates the percentages of inhibition of the multiplication of the amastigotes after 30 minutes of contact with the serum of the immunized dog (ESP), relative to the serum of the healthy dog:

| % inhibition | 1^{st} day | 2^{nd} day | 3^{rd} day | Average |
|---|---|---|---|---|
| Ip/Sp | 66% | 68% | 61% | 65% |
| I ½/S ½ | 47% | 60% | 42% | 51% |
| I ¼/S ¼ | 24% | 47% | 47% | 39% |
| I ⅛/S ⅛ | 15% | 18% | 24% | 19% |

Interpretations:

An inhibition of the growth is noticed on the kinetics of growth of amastigotes when before differentiation, the promastigotes have been put in contact with an anti-ESP serum. The growth of amastigotes is then slowed down and this in a manner proportional to the concentration of serum. The more the serum of the dog immunized to ESP is concentrated, the more the multiplication of the arnastigotes is inhibited. Thus, there is a dosage-dependence effect.

3. Results Obtained in other Dogs: Dogs Immunized with ESP and Protected Against Experimental Infection and Natural Infection.

The average viability of the promastigotes after contact with the serum of the dog immunized vaccine complex ESP is 16.8% (5 dogs vaccinated) and 72.9% (5 dogs placebo) for serum of non-immunized dogs. Also, antibodies of the immunized dog not linked to the complement are cytotoxic for the promastigotes.

The following table indicates the percentages of viability of promastigotes after contact, with the serums of immunized dogs and non-immunized dogs with ESP.

| | Dogs analyzed | | | | | | | |
|---|---|---|---|---|---|---|---|---|
| | | | Experimental Studies (experimental infection) | | | | | |
| | T1* | T2* | E | O | G | K | 7 | 16 |
| | (control) | (control) | Placebo | Placebo | Vaccine | Vaccine | Vaccine | Placebo |
| Percentage | 100% | 100% | 85.7% | 61.9% | 22.4% | 10.1% | 12.8% | 76.2% |
| viability | | | | | | | | |

| | Dogs analyzed | | | | | |
|---|---|---|---|---|---|---|
| | | | Studies in local environment (natural infection) | | | |
| | T1* | T2* | HC 046 | HC 051 | LB 120 | RR 019 |
| | (control) | (control) | Placebo | Vaccine | Placebo | Vaccine |
| Percentage | 100% | 100% | 66.7% | 15.7% | 73.8% | 22.9% |
| viability | | | | | | |

Dogs T1 and T2: healthy non-immunized dogs which did not receive the placebo.

4. Correlation between Production of IgG₂ Anti ESP and the Effect of the Serums on the Viability of the Promastigotes

According to the table below (Correlation IgG2 anti ESP/inhibition proliferation of the promastigotes), the fall in the percentage of viability of the promastigotes corresponds to the presence of IgG2 specific to the vaccine complex ESP.

| | Dogs experimentally infected | | | | | | | |
|---|---|---|---|---|---|---|---|---|
| | MINON | MUMA | E | O | G | K | 7 | 16 |
| IgG total By IF* | — | — | — | — | — | — | — | — |
| IgG2 specific to | 0.391 | 0.108 | 0.065 | 0.104 | 0.299 | 0.299 | 0.310 | 0.056 |
| ESP** by ELISA | | | | | | | | |
| Percentage viability | 50% | 100% | 85.7% | 61.9% | 22.4% | 10.1% | 12.8% | 76.2% |

Dogs naturally infected

| | HC 046 | HC 051 | LB 120 | RR 019 |
|---|---|---|---|---|
| IgG total By IF* | — | — | — | — |
| IgG2 specific to | 0.054 | 0.321 | 0.065 | 0.386 |
| ESP** by ELISA | | | | |
| Percentage viability | 66.7% | 15.7% | 73.8% | 22.9% |

*IF: Immunofluorescence: negative: rate ≦ 1/100
**IgG₂: DO at 492 nm: the numbers in boldface correspond to positive results

5. Study of the IgG2 in Leishmanian Dogs Treated by Chemotherapy.

The 5 leishmanian dogs had, at the start of treatment, a higher rate oftotal IgG (≧1/200), this rate stayed fixed or dropped to 1/50 4 months after the start of treatment for 4 dogs.

The fifth one died 2 months after treatment, he had an increase in total IgG (1/200 to 1/800) and was negative in IgG₂ directed against the carboxyterminal part of PSA.

On the contrary, the 4 other dogs recovered and have IgG₂ against the carboxyterminal part of the PSA.

6. Study of IgG₂ in Leishmanian Dogs Treated by Immunotherapy with ESP:

The three dogs have at the start of treatment a very high rate of total IgG. This rate decreases 3 months after treatment. In parallel to this drop in total IgG, the clinical signs become unclear and IgG2s specific to the product injected appear (see table below)

| | | Leishmanian dogs | | |
|---|---|---|---|---|
| | | LAFAYETTE | JERRY | RODEO |
| IgG total | Before | 1/800 | 1/3200 | 1/800 |
| by IF* | treatment | | | |
| | After | 1/200 | 1/400 | 1/100 |
| | treatment | | | |
| IgG specific | Before | 0.052 | 0.060 | 0.059 |
| to ESP by | treatment | | | |
| ELISA** | | | | |
| | After | 0.361 | 0.327 | 0.359 |
| | treatment | | | |

*IF: Immunofluorescence: negative rate ≦ 1/100
**IgG2: DO at 492 nm, the underlined characters correspond to positive results.

It thus appears that the capacity to produce the IgG₂ antibodies against this epitope specific to PSA accompanies the evolution towards recovery of the dogs. Thus, it is indeed the IgG₂s which are responsible for the lysis of the amastigotes and promastigotes of Leishmania sp in vitro and for the neutralization of their proliferation.

The analysis of the serums of 3 leishmanian dogs treated by the ESP vaccine complex gives exactly the same type of result, i.e. an appearance of IgG₂s specific (to the carboxyterminal part of PSA of the ESP) correlating with an activity of the serum neutralizing the proliferation of the promastigotes of Leishmania infantum in vitro and a cell-mediated immunity of Th1 type.

These IgG₂, in dogs, or other particular isotypes in the other mammals associated with a cellular immunity of the Th1 type, can be detected by various in vitro methods, for example: ELISA, DOT BLOT, WESTERN BLOT, INOCHROMATOGRAPHY, IMMUNO NEPHELOMETRY, RIA, IMMUNO PRECIPITATION, ELECTRO SYNERESIS and any other in vitro method that causes intervention of a conjugated system or other systems of visualization for the Ag—Ac reaction. Thus, the immunoglobulins according to the invention can be used for a diagnostic product that makes it possible to detect one or more epitopes carried by the terminal ends NH₂ and COOH of the Protein Surface Antigen excreted-secreted by Leishmania sp. For example, an ELISA system can be used on a plastic support and a WESTERN BLOT system on membranes of nitrocellulose or other polymers that cause an enzymatic conjugate to intervene. Latex supports can also be used. Some radio-isotopes, fluorescent molecules, luminescent molecules or particles of color can be coupled to these various antigens to be used as conjugates. Some metallic colloids can also be used. In addition, excretion-secretion antigens of amastigotes or promastigotes of Leishmania sp and more particularly the carboxyterminal part of the Protein Surface Antigen in purified form or recombinant protein form are coupled to the large molecules of biotin, avidin, streptavidine or any other protein to make them more accessible.

Thus, antibodies of the isotype IgG2 and the corresponding sub-classes according to the invention can be used in mammals as markers of an immunitary state of the cell-mediated type depending on T lymphocytes and preferably the T lymphocytes of the Th1 type, like markers of the resistance to leishmaniasis and to infections by pathogenic intracellular micro-organisms in mammals, as markers of the immunoprophylactic and immunotherapeutic vaccination for infections by pathogenic intracellular micro-organisms, and as effectors of immunotherapy for leishmaniases and infections by pathogenic intracellular micro-organisms.

1. Immunoglobulins characterized in that they are immunoglobulins of the classes IgG₂ and corresponding sub-classes, specific to the excretion-secretion antigens of promastigotes or amastigotes of Leishmania sp, capable of lyzing the amastigotes and promastigotes of Leishmania sp in vitro and neutralizing their proliferation.

2. Immunoglobulins according to claim 1, characterized in that they are specific to the major immunogen, excreted-secreted by promastigotes or amastigotes of Leishmania sp, belonging to the family of the Protein Surface Antigens and corresponding to a range of molecular mass from 52 to 58 Kda.

3. Immunoglobulins according to claim 2, characterized in that they are specific to the carboxyterminal part of the major excreted-secreted immunogen.

4. Immunoglobulins according to claim 1, characterized in that they are isotypes IgG₂ in dogs and specific isotypes in other mammals, isotypes linked to cell-mediated immunity depending on T lymphocytes of the Th1 type.

5. Use of immunoglobulins according to claim 1 as markers of a cell-mediated immunity allowing notably the detection of a cell-mediated immunity depending on T lymphocytes and preferably T lymphocytes of the Th1 type in mammals.

6. Use of immunoglobulins according to claim 1 as markers of the resistance to leishmaniasis and to infections by pathogenic intracellular micro-organisms in mammals.

7. Use of immunoglobulins according to claim 1 as markers of immunoprophylactic and immunotherapeutic vaccination in mammals for leishmaniases and infections by pathogenic intracellular micro-organisms.

8. Immunoglobulins according to claim 1, as effectors of immunotherapy in the context of leishmaniases and infections by pathogenic intracellular micro-organisms in mammals.

9. Use of immunoglobulins according to claim 1, for an in vitro diagnostic product detecting one or more epitopes carried by the terminal ends NH₂ and COOH of the Protein Surface Antigens excreted-secreted by Leishmania sp.
